(19) United States
(12) Patent Application Publication
(10) Pub. No.: US 2024/0249843 A1
(43) Pub. Date: Jul. 25, 2024

(54) PRECISION MEDICINE SYSTEMS AND METHODS

(71) Applicant: TMAccelerator Company, LLC, Boston, MA (US)

(72) Inventors: Jose FERNANDEZ, Cambridge, MA (US); Lina WILLIAMSON, Pittsboro, NC (US); Joshua RESNIKOFF, Harwich, MA (US)

(21) Appl. No.: 18/560,635

(22) PCT Filed: May 16, 2022
(86) PCT No.: PCT/US22/72352

§ 371 Date: Nov. 13, 2023

Related U.S. Application Data
(60) Provisional application No. 63/201,828, filed on May 14, 2021.

(51) Int. Cl. G16H 50/20; G16H 10/40; G16H 10/60; G16H 15/00; G16H 20/00; G16H 50/70

(52) U.S. Cl. CPC G16H 50/20; G16H 10/40; G16H 10/60; G16H 15/00; G16H 20/00; G16H 50/70

(57) ABSTRACT

A precision medicine system for patient management and rare disease identification can include an interface and a global data lake. The interface can be configured to search a plurality of search databases including medical data related to diagnosis or treatment plans. The global data lake can be configured to include a plurality of patient data lakes each storing patient data of at least one patient. The precision medicine system can further include a processor and a memory configured to label the patient data using medical ontologies and selectively produce relevant medical data from the search databases via the interface. The patient data can then be supplemented with the medical data. Embodiments enable clinicians to provide specialized, up to date diagnosis and treatment plants to patients by providing the clinicians with medical advancements specific to their patient.

### CROSS-REFERENCE TO RELATED APPLICATION

This application claims the benefit of U.S. Provisional Application No. 63/201,828, filed May 14, 2021, the disclosure of which is hereby incorporated by reference herein.

### TECHNICAL FIELD

Embodiments of the present disclosure relate generally to the field of patient management, and more particularly to coordinating patient medical histories for rare-disease identification.

### BACKGROUND

The current healthcare environment provides inadequate methods for clinicians to obtain, process, and maintain a patient's complete medical history. The current healthcare system fails to extract important and needed knowledge from one of its most important work products—patients' treatment records. That is, millions of patients are treated and cared for each year, but the demographics, epidemiology, and records of patients' diseases, as well as the outcomes from treatment, are kept in a form that prevents general application of this information.

Further, the current healthcare environment lacks the ability to keep physicians up to date with respect to the latest developments in medical science. This includes developments in disease diagnosis and treatment in highly specific instances, such as rare diseases. A generally accepted definition for classifying rare diseases is if the disease affects not more than one patient in two thousand. This standard can vary depending on the healthcare system used and can be narrower, such as not more than one patient in five thousand cases. To help address these varied definitions, certain ontologies for classifying diseases, such as the MONDO DISEASE ONTOLOGY (Mondo), aim to harmonize disease definitions across the world by providing logic-based structures for unifying multiple disease resources. Any disease term in Mondo is considered rare if the term, or its ancestor, has modifier MONDO:0021136 ‘Rare’ in the ontology. Applying this definition there are nearly 13,000 Mondo rare disease terms. Further compounding this issue is the exponentially increasing number of new methods for diagnosis and treatment of disease. Moreover, the obsolescence of medical information is so rapid that the information that a physician learned in formal medical training is often no longer state-of-the-art medicine after several years in the field.

When determining a treatment plan for a patient, physicians often attempt to apply a standard of care. Such informal or formal guidelines that are generally accepted in the medical community for the treatment of a disease or condition. Standards of care, as discussed herein, may be developed by a specialist societies or organizations and the title of standard of care awarded at their own discretion. Each standard of care can be a clinical practice guideline, a formal diagnostic and treatment process a healthcare provider will follow for a patient with a certain set of symptoms or a specific illness. That standard will follow guidelines and protocols that experts would agree with as most appropriate, also called “best practice.” Standards of care are developed in a number of ways; sometimes they are simply developed over time, and in other cases, they are the result of clinical trial findings

Physicians must increasingly know intricate details about diseases and the most up to date treatment regimens for such diseases. This includes knowledge of highly specialized areas of medicine and being able to quickly diagnose conditions within these areas based on observed and reported symptoms. It also may be directed to areas which are not a focus of a particular clinician's medical practice, potentially leading to a misdiagnosis of a patient's symptoms. A misdiagnosis can exacerbate a patient's condition and the onset of disabilities or comorbidities.

Accordingly, there exists a need for methods which can aid clinicians in processing a patient's potentially extensive medical history while providing relevant diagnosis and treatment procedures.

Further, rare and complex diseases are often poorly understood and little advancement can be made until the “signature” of the disease (molecular, genetic, environmental characteristics, etc.) is known. Locating enough patients exhibiting a rare disease can be extremely difficult, preventing development of pharmaceuticals and treatment plans to improve the treatments available for these patients. Therefore, a method of identifying and aggregating rare disease patients is needed.

There have been attempts to solve some of the problems discussed. There have been computer-based methods for acquiring and storing health information from patients. For example, U.S. Pat. No. 10,276,261 discloses that a patient's medical history can be acquired via a template, compared to predetermined criteria for confirming a diagnosis, and stored in a database using computer technology. However, this system relies heavily on manual input and judgment from a contributor, such as a health care provider, and does not automatically suggest alternative diagnosis, conveniently present advances in medical research, or the like.

Another computer-based system is described in U.S. Pat. No. 7,991,485 that discloses a computer-implemented method for acquiring and evaluating patient information. This method, however, requires manual entry of medical history information by a patient or physician, does not automatically incorporate relevant up to date medical research on a patient specific basis, dynamically develop diagnosis and treatment plans based on information learned during monitoring, or the like.

### SUMMARY

Embodiments of the present disclosure provide a system for coordinating patient medical histories to identify rare diseases. In embodiments, the system allows a user to ingest patient medical histories, research relevant diagnosis and treatment plans, and associate similar patient medical histories into cohorts.

In one aspect of the present disclosure, a system for coordinating patient medical histories for rare-disease identification comprises an interface configured to search a plurality of search databases including medical data and a global data lake configured to include a plurality of patient data lakes each patient data lake can be configured to store patient data of at least one patient of a plurality of patients. The system can further include at least one processor having a memory operably coupled to the processor.

The memory further comprises instructions that, when executed on the at least one processor, cause the at least one processor to for each patient of the plurality of patients, receive a medical history for the patient comprising at least one record. Each record of the medical history can be labelled according to at least one medical ontology. The processor can generate a plurality of search paths based on the labeled records and label the plurality of search paths according to the at least one medical ontology. The processor can identify commonalities between the plurality of search paths, identify pairs of search paths of the plurality of search paths that are mutually exclusive, and selectively request medical data relevant to the patient based on the identified commonalities between the plurality of search paths and the identified pairs of the plurality of search paths that are mutually exclusive from at least one of the plurality of search databases. The processor can update the patient data to include the medical data.

In embodiments, at least one of the search paths includes a symptom of the patient and the medical data is a condition corresponding to the symptom of the patient. In embodiments, at least one of the search paths includes a diagnosis of the patient and the medical data is a treatment associated with the diagnosis of the patient.

In embodiments, the instructions are further configured to cause the processor to identify at least one item of medical information selected to differentiate the plurality of search paths that are mutually exclusive, and in response to receiving the at least one item of medical information, selecting a search path of the search paths that are mutually exclusive and requesting the medical data from at least one of the plurality of search databases based on the selected search path.

In embodiments, the at least one item of medical information is a result of a medical test. The medical test can be a test to determine whether a gene is expressed in the patient.

In embodiments, the instructions are further configured to cause the processor to, for each patient of the plurality of patients, augment at least one record of the medical history by requesting medical data relevant to the at least one record from at least one of the plurality of search databases.

In embodiments, the instructions are further configured to cause the processor to, for each patient of the plurality of patients, identify at least one label associated with a condition of the patient, identify at least one label associated with a treatment of the patient, determine a plurality of expected treatments associated with the condition by requesting medical data relevant to the at least one label associated with the condition of the patient from at least one of the plurality of search databases, and produce an output indicating any expected treatments of the plurality of expected treatments that do not have at least one label associated with a treatment of the patient.

In embodiments, the output can further indicate whether the treatment of the patient is associated with at least one expected treatment of the plurality of expected treatments.

In embodiments, the instructions are further configured to cause the processor to selectively request medical data relevant to the patient based on the plurality of search paths from at least one second patient data lake of the plurality of patient data lakes.

In embodiments, at least one of the plurality of search databases can be periodically monitored for updated medical data.

In one aspect of the present disclosure, a computer-implemented method for coordinating patient medical histories for rare-disease identification includes receiving, at a computing device comprising a memory and a processor, a medical history for a patient comprising at least one record, labelling each record of the medical history according to at least one medical ontology, storing each labeled record in a patient data lake configured to electronically store patient data of the patient, generating a plurality of search paths based on the labeled records, labelling each search path of the plurality of search paths according to the at least one medical ontology, identifying commonalities between the plurality of search paths, identifying pairs of the plurality of search paths that are mutually exclusive, selectively requesting medical data relevant to the patient based on the identified commonalities between the plurality of search paths and the identified pairs of the plurality of search paths from at least one search database communicably coupled to the computing device, and updating the patient data lake to include the medical data.

In one aspect of the present disclosure, a method for consolidating and presenting a medical history of a patient includes receiving the medical history of the patient, the medical history including target patient data, labeling, via a natural language processing system, the target patient data based on medical ontologies, storing the target patient data in one of a plurality of patient data lakes, comparing the target patient data with existing patient data from at least one of the plurality of patient data lakes to identify similarities based on at least one of symptoms, diagnosis, or treatment plans, classifying a relevance of the target patient data based on the labels and the identified similarities; and producing a report of the target patient data classified as most relevant.

In one aspect of the present disclosure, a system for associating patients comprises a global data lake configured to include a plurality of patient data lakes each storing patient data of at least one patient, at least one processor having a memory operably coupled to the processor; and the memory further comprising instructions that, when executed on the at least one processor, cause the at least one processor to, for each of the plurality of patients: label the patient data using natural language processing based on medical ontologies, generate a diagnosis report confirming a previous diagnosis or providing a first alternative diagnosis, receive diagnosis feedback from a user, the diagnosis feedback including a selected diagnosis, generate a treatment plan based on the selected diagnosis, receive treatment plan feedback from the user, the treatment plan feedback including monitored patient outcomes as a result of the treatment plan, update the diagnosis report to the selected diagnosis or provide a second alternative diagnosis based on the labeled patient data, the diagnosis feedback, and the treatment plan feedback.

In one aspect of the present disclosure, a method for consolidating and presenting a medical history of a patient comprises receiving the medical history of the patient, the medical history including target patient data, labeling, via a natural language processing system, the target patient data based on medical ontologies, storing the target patient data in one of a plurality of patient data lakes, comparing the target patient data with existing patient data from at least one of the plurality of patient data lakes to identify similarities based on at least one of symptoms, diagnosis, or treatment plans, classifying the relevance of the target patient data based on the labels and the identified similarities, and producing a report of the target patient data classified as most relevant.

In one aspect of the present disclosure, a method for confirming a diagnosis of a patient can include receiving diagnosis feedback from a user, including a diagnosis that was ultimately made, generating a treatment plan based on the diagnosis feedback, receiving treatment plan feedback from the user that details monitored patient outcomes and associating the patient with a patient cohort based on the labeled patient data, diagnosis feedback, and treatment plan feedback.

In another aspect of the present disclosure, a method for consolidating and presenting a medical history of a patient is described. The method can comprise receiving the medical history of the patient, including target patient data, labeling the target patient data based on medical ontologies via a natural language processing system, and storing the target patient data in one of a plurality of patient data lakes. The method can further comprise comparing the target patient data with existing patient data from at least one of the plurality of patient data lakes to identify commonalities based on at least one of symptoms, diagnosis, or treatment plans. The relevance of the target patient data can then be classified based on the assigned labels and identified commonalities. Lastly, a report may be produced, presenting the most relevant patient data.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIG. 1 is a block diagram depicting a system for managing a global patient data lake, according to an embodiment.

FIG. 2 is a block depicting a global data lake management engine of the system of FIG. 1, according to an embodiment.

FIG. 3 is a flowchart depicting transitions between patient intake stages, according to an embodiment.

FIG. 4 is a flowchart depicting an automated search process for retrieving relevant documentation from multiple data stores, according to an embodiment.

FIG. 5 is a block diagram depicting an update process, according to an embodiment.

FIGS. 6A, 6B, 6C, and 6D are representations of user interfaces, according to embodiments.

FIG. 7 is a flowchart depicting a method for displaying patient data, according to an embodiment.

FIG. 8 is a flowchart depicting a method for classifying a patient's medical history, according to an embodiment.

FIGS. 9A and 9B are representations of an example clinical history document.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION OF THE DRAWINGS

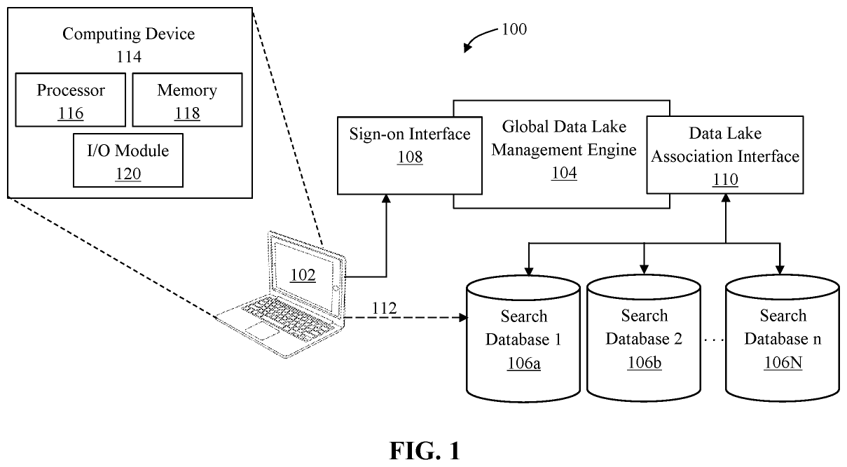

Referring to FIG. 1, a block diagram depicting a system 100 for managing a global patient data lake is depicted, according to an embodiment of the present disclosure. System 100 comprises at least one user device 102, global data lake management engine 104, and plurality of search databases 106a-106N.

In embodiments, system 100 and/or its components or subsystems can include computing devices, microprocessors, modules and other computer or computing devices, which can be any programmable device that accepts digital data as input, is configured to process the input according to instructions or algorithms and provides results as outputs. In one embodiment, computing and other such devices discussed herein can be, comprise, contain, or be coupled to a central processing unit (CPU) configured to carry out the instructions of a computer program. Computing and other such devices discussed herein are therefore configured to perform basic arithmetical, logical, and input/output operations.

Computing and other devices discussed herein can include memory. Memory can comprise volatile or non-volatile memory as required by the coupled computing device or processor to not only provide space to execute the instructions or algorithms, but to provide the space to store the instructions themselves. In one embodiment, volatile memory can include random access memory (RAM), dynamic random-access memory (DRAM), or static random-access memory (SRAM), for example. In one embodiment, non-volatile memory can include read-only memory, flash memory, ferroelectric RAM, hard disk, floppy disk, magnetic tape, or optical disc storage, for example. The foregoing lists in no way limit the type of memory that can be used, as these embodiments are given only by way of example and are not intended to limit the scope of the disclosure.

In embodiments, the system or components thereof can comprise or include various modules or engines, each of which is constructed, programmed, configured, or otherwise adapted to autonomously carry out a function or set of functions. The term “engine” as used herein is defined as a real-world device, component, or arrangement of components implemented using hardware, such as by an application specific integrated circuit (ASIC) or field programmable gate array (FPGA), for example, or as a combination of hardware and software, such as by a microprocessor system and a set of program instructions that adapt the engine to implement the particular functionality, which (while being executed) transform the microprocessor system into a special-purpose device. An engine can also be implemented as a combination of the two, with certain functions facilitated by hardware alone, and other functions facilitated by a combination of hardware and software. In certain implementations, at least a portion, and in some cases, all, of an engine can be executed on the processor(s) of one or more computing platforms that are made up of hardware (e.g., one or more processors, data storage devices such as memory or drive storage, input/output facilities such as network interface devices, video devices, keyboard, mouse or touchscreen devices, etc.) that execute an operating system, system programs, and application programs, while also implementing the engine using multitasking, multithreading, distributed (e.g., cluster, peer-peer, cloud, etc.) processing where appropriate, or other such techniques. Accordingly, each engine can be realized in a variety of physically realizable configurations, and should generally not be limited to any particular implementation exemplified herein, unless such limitations are expressly called out. In addition, an engine can itself be composed of more than one sub-engines, each of which can be regarded as an engine in its own right. Moreover, in the embodiments described herein, each of the various engines corresponds to a defined autonomous functionality; however, it should be understood that in other contemplated embodiments, each functionality can be distributed to more than one engine. Likewise, in other contemplated embodiments, multiple defined functionalities may be implemented by a single engine that performs those multiple functions, possibly alongside other functions, or distributed differently among a set of engines than specifically illustrated in the examples herein.

In embodiments, user device 102 can be computing device 114 comprising processor 116, memory 118, and input/output module 120. In embodiments, user device 102 can comprise a smartphone, tablet computer, laptop computer, desktop computer, server, virtual machine, or the like. Input/output module 120 can comprise user interfaces and communication systems enabling communication with other computing devices 114, components of system 100, or other computing systems. Communication systems can comprise wired or wireless network interface devices and the like.

In embodiments, user device 102 can present a plurality of user interfaces, types such as text-based, graphical user interface (GUI), three-dimensional (3D) or other interface components. User device 102 can comprise or interface with one or more input mechanisms such as a keyboard, mouse, touchpad, joystick, or any device capable of receiving user input known in the art. User device 102 can comprise or interface with one or more output mechanisms such as a display screen, projector, audio output, or any other dynamic output mechanism known in the art. In embodiments, user device 102 can comprise a programmatic interface such as an application programming interface (API), enabling programmatic interaction with system 100 for data entry, data retrieval, configuration, or any other user task.

User device 102 can be used to access one or more patient profiles from global data lake management engine 104 such that patient information pertaining to the patient profile is selectively provided through sign-on interface 108. In embodiments, user device 102 is configured to communicate sign-on information to global data lake management engine 104. Sign-on information can be used to restrict access to certain users and allow for multiple layers of security. Further, sign-on information permits all access and edits of a user to be logged for security and tracking purposes.

In embodiments, user device 102 can be used to provide new patient, chosen treatment plans, or other data to global data lake management engine 104. Global data lake management engine 104 can additionally access the plurality of search databases 106 through data lake association interface 110. In embodiments, global data lake management engine 104 can associate certain patient data with search databases 106 that are particularly relevant to the patient's symptoms or medical history. Data lake association interface 110 facilitates communication between global data lake management engine 104 and search databases 106, including sending search requests to search databases 106 and processing data returned from the search such that incoming data can be incorporated into global data lake management engine.

Search databases 106 can include data sources in one or more domains such as diagnosis, medications, genetics, or the like. Search databases 106 can further comprise repositories of clinical practices guidelines and/or sources of standards of care. Search databases 106 can comprise knowledge graphs, ontologies, controlled vocabularies, or other data stores providing information about relationships between medical concepts. For example, in embodiments, search databases 106a-N can comprise publicly available databases such as DISEASE ONTOLOGY, GENECARDS, MALACARDS, GOOGLE SCHOLAR, CLINICALTRIALS.GOV, and the like.

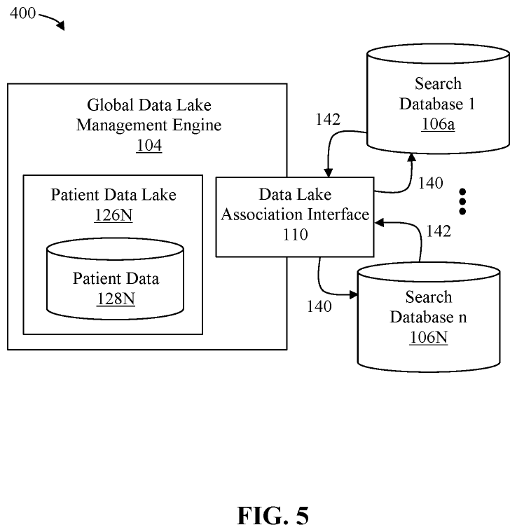

System 100 facilitates a continually up to date connection between user device 102, global data lake management engine 104, and search databases 106. User access to medical information, including patient medical histories stored within global data management engine 104 can be securely monitored. Further, the connection between global data lake management engine 104 and search databases 106 facilitates an update process as depicted in FIG. 5 and described in detail below.

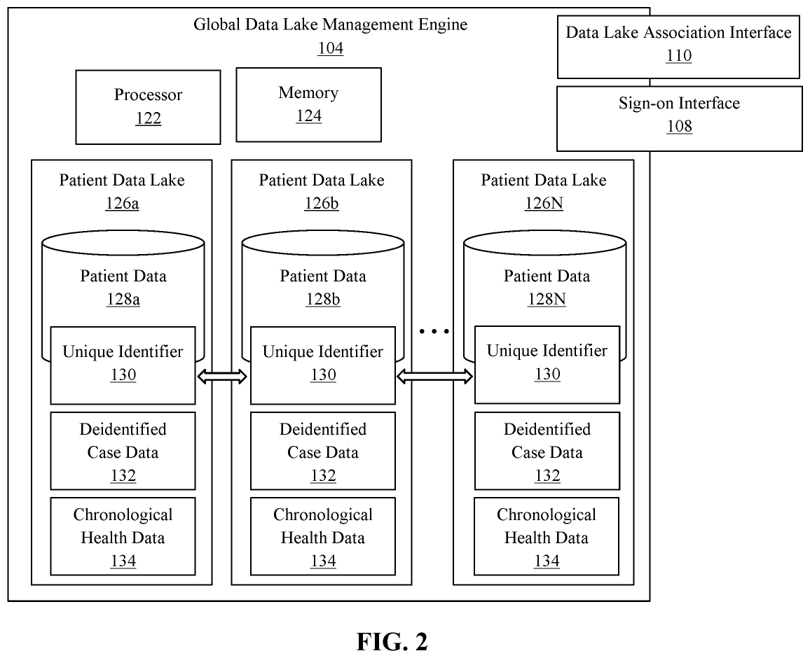

Referring to FIG. 2, a block diagram of global data lake management engine 104 of system 100 is further depicted, according to an embodiment. Global data lake management engine 104 can comprise processor 122, memory 124, and patient data lakes 126a-126N. In embodiments, global data lake management engine 104 can comprise a global data lake including a plurality of patient data lakes. In other embodiments, global data lake management engine 104 acts as a global data lake and controls the conveyance of data to patient data lakes 126.

As described above, processor 122 can include fixed function circuitry and/or programmable processing circuitry. Processor 122 can include any one or more of a microprocessor, a controller, a DSP, an ASIC, an FPGA, or equivalent discrete or analog logic circuitry. In some examples, processor 122 can include multiple components, such as any combination of one or more microprocessors, one or more controllers, one or more DSPs, one or more ASICs, or one or more FPGAs, as well as other discrete or integrated logic circuitry. The functions attributed to processor 122 herein may be embodied as software, firmware, hardware or any combination thereof.

Memory 124 can include computer-readable instructions that, when executed by processor 122 cause global data lake management engine 104 to perform various functions. Memory 124 can include can volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media.

In embodiments, global data lake management engine 104 can comprise a plurality of patient data lakes 126a-126N. Each patient data lake can be configured to store patient data 128a-128N for patients within system 100. Data lakes can comprise data stores, databases, file systems, memories, or other storage systems appropriate to store and provide the described data items. Global data lake and patient data lakes 126 can store all types of data: structured, semi-structured, or unstructured. Each data lake can have one or more associated abstract or concrete knowledge graphs and maps thereof to enable navigation of data items stored therein. Data lakes can comprise object-relational mapping (ORM) storage systems and associated APIs. Global data lakes and patient data lakes 126 described herein can reside within a single database system or across a plurality of database systems. Database systems used in implementations can include Apache Hadoop, Hadoop Distributed File System (HDFS), Microsoft SQL Server, Oracle, Apache Cassandra, MySQL, MongoDB, MariaDB or the like.

Each data store can comprise logical groupings of data. Numerous types and structures of data can be stored and indexed. Where, as depicted or described, data structures are said to include or be associated with other data structures, it should be understood that such other data structures may be stored within or in association with each data structure, or may be referenced by other data structures through the use of links, pointers, or addresses, or other forms of referencing data.

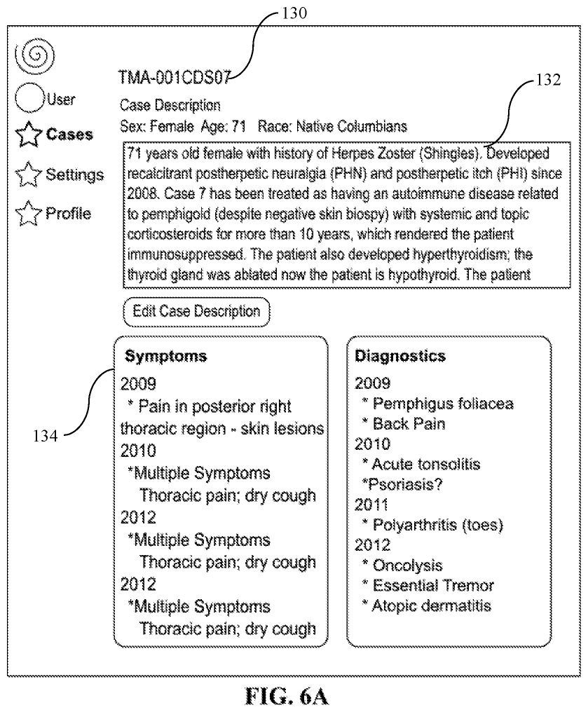

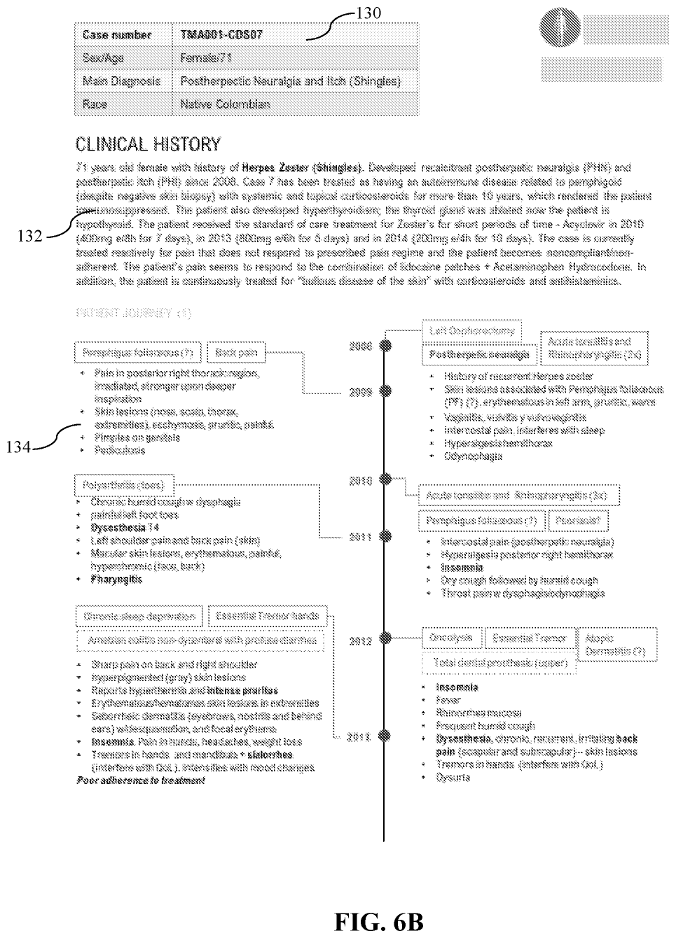

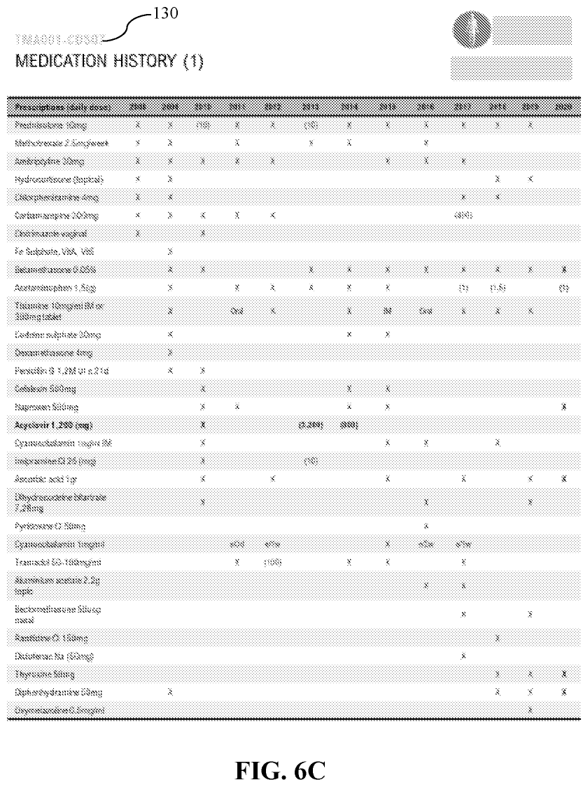

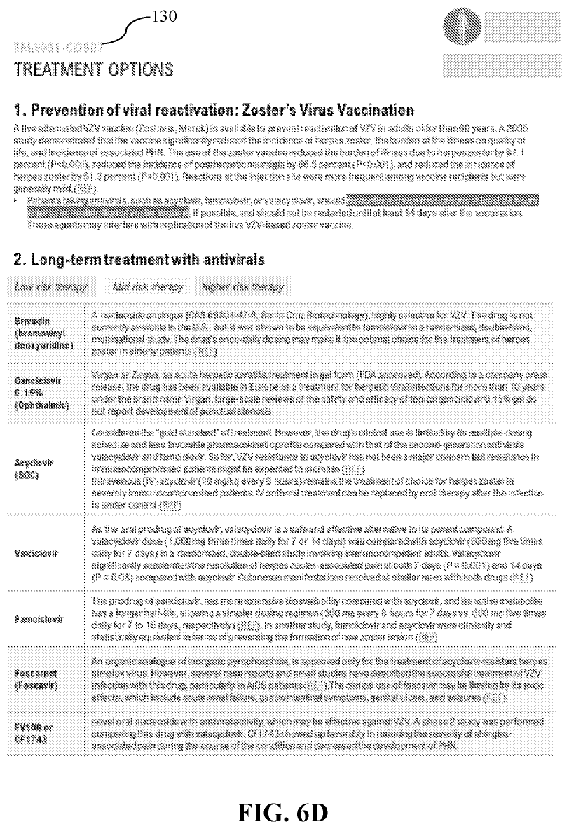

Patient data lakes 126 within global data lake management engine 104 can be configured to store patient data 128 for patients within system 100. Patient data 128 can comprise a series of records including a unique identifier 130, deidentified case data 132, and patient medical history 134. In embodiments, unique identifier 130, deidentified case data 132, and patient medical history 134 can be used in report generation and displayed on user interfaces, as represented in FIGS. 6A-6D.

Patient data 128 can comprise a plurality of records (which may also be referred to as elements, items, or fields). Each patient data record can have one or more associated labels. elements corresponding to a label or clinical named entity according to one or more medical ontologies or vocabularies. Clinical named entities can correspond to symptoms, phenotypes, conditions, diseases, diagnoses, treatments (such as procedures or medications), or combinations thereof. Labelling can be based on medical ontologies, such as ICD-10-CM, RXNorm, Chemical Entities of Biological Interest (ChEBI), and the like. Each label can comprise a concept in one or more ontologies or controlled vocabularies. Each label can be stored or represented as a Compact Uniform Resource Identifier (CURIE) in the form of <knowledge-source>:<id> such as DOID:3364 for “migraine.” In embodiments, labels associated with patient data 128 for each patient can be used to better group the patient in patient cohorts.

In embodiments, a patient data lake can comprise patient data for multiple patients or a cohort of similar patients. Further, while patient data lakes 126a-126N are depicted, it should be understood that global data lake management engine 104 can be configured to store any number of patient data lakes 126a-126N and corresponding patient data 128a-128N.

In embodiments, each patient is assigned a unique identifier 130 that is used to identify a case record without associating the case record with personally identifiable information (PII). PII can include information such as a user's name, birthdate, government issued identification (ID) number, financial account information, or any other data that could potentially identify a specific individual. PII data may be defined by one or more regulations or standards regarding the protection of PII. The separation, and in some cases outright removal, of PII from patient data 128 can facilitate compliance with the Health Insurance Portability and Accountability Act (HIPAA), General Data Protection Regulation (GDPR), and other regulatory standards directed to data protection and privacy.

In embodiments, deidentified case data 132 can comprise a PII-free record of any recent diagnosis and medical history of the patient. Deidentified case data 132 can provide a high-level overview of a patient's recent status for a clinician to review. Deidentified case data 132 can include patient compliance and outcomes from recent treatment plans to quickly determine if a previous diagnosis and determined treatment plan are effective.

In embodiments, patient medical history 134 can comprise electronic medical record (EMR) consolidation and visualization. Patient medical history 134 can comprise a complete life history incorporating long term patient data. Patient medical history 134 can comprise information about allergies, illnesses, surgeries, immunizations, results of physical exams and tests, and other information related to medical care. A patient's medical history may also include information about medicines taken and health habits, such as diet and exercise.

For example, patient medical history 134 can comprise medication history including duration of medicine, prescription details, whether a brand or generic name medicine was used, and the quantity and dosing regimen for the medicine. A patient's medication history may be further enhanced by hover boxes capable of showing symptoms the medication was administered to treat. In embodiments, chronological health data can be color coded to represent indications for known adverse effects associated with past medication or treatment plans. In such embodiments, a clinician may be more likely to determine if any current symptoms are the result of prior treatments. In embodiments, patient medical history 134 can be segmented visually in a timeline, such as by year, and include linkable keywords to medical research or other documentation for further clinician research if desired.

Global data lake management engine 104 is capable of labeling and organizing a plurality of patient data lakes to facilitate fast and efficient access to patient data. Additionally, associations between various patient data lakes 126a-126N can be made based on commonalities between them. Data lake association interface 110 can process incoming distilled data from search databases before storing the distilled data in relevant patient data lakes 126a-126N. These features are an improvement over conventional systems that cannot efficiently relate and compare large quantities of patient data or are incapable of continually supplementing patient data with up to date medical research pertinent to individual patients' symptoms, diagnosis, and treatment plan.

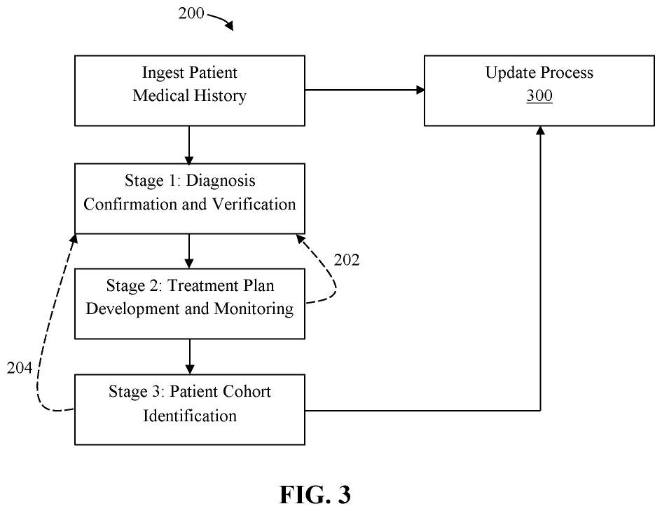

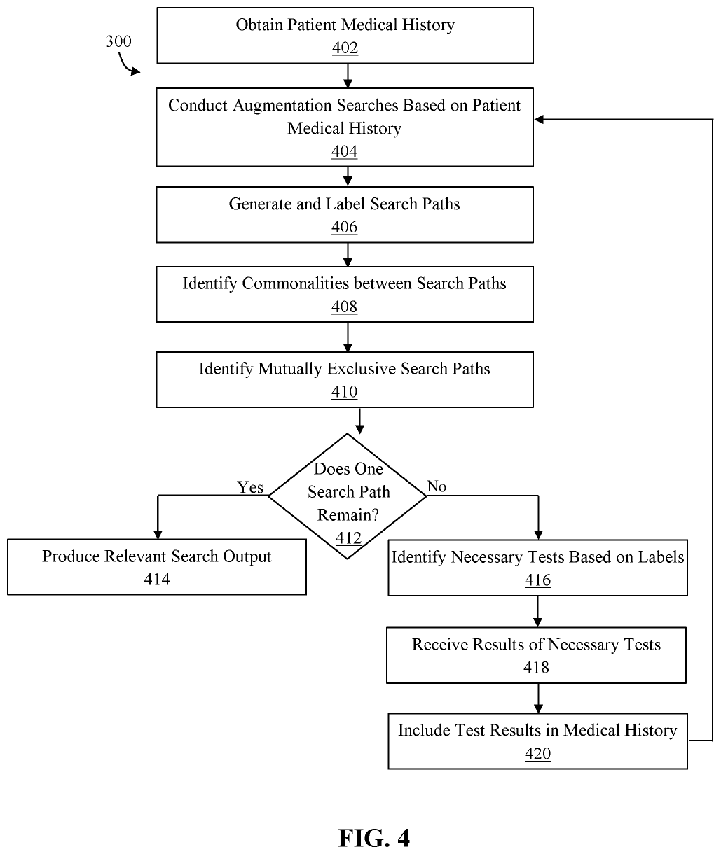

Referring to FIG. 3, a flow chart of patient data management process 200 is depicted according to an embodiment. Patient data management process 200 can generally comprise three stages of processing a patient's medical record or medical history. It should be understood that process 200 as depicted and described is an example and embodiments can include more, fewer, or alternative stages. Additionally, the individual stages or steps used in process 200 may be performed in any order and/or simultaneously, as long as the teaching remains operable. Before these stages, the patient's medical history can be automatically deidentified, processed, and stored to a patient data lake using a natural language processing (NLP) system. Each new update to patient data triggers update process 300 as shown in FIG. 4 and described below.

### Stage 1: Diagnosis Confirmation and Verification

Stage 1 of patient intake process 200 comprises diagnosis confirmation and verification. During this stage the diagnosis is confirmed or the clinician is given alternative diagnoses to consider. To determine the accuracy of a diagnosis and to generate alternative diagnosis, the global data lake is searched for similar symptoms and diagnoses based on the patient's medical history. Once the search is complete, a report is provided to the clinician and clinician feedback is received. In embodiments, clinician feedback can be considered when making future diagnosis decisions for patients with similar medical histories. A primary goal of stage 1 is to achieve a more precise diagnosis, such as taking an initial diagnosis of epilepsy and narrowing the original diagnosis to a diagnosis of early infantile epileptic encephalopathy (EIEE).

### Stage 2: Treatment Plan Development and Monitoring

Stage 2 of patient intake process 200 comprises presenting suggested treatments plans based on the determined diagnosis. Widely adopted treatment plans can be automatically presented to the clinician based on diagnosis. Any treatment plan suggestions presented to the clinician can be edited before approval. After a treatment plan has been developed patient outcomes are monitored and results are stored back into the system. Information learned during monitoring can result in changing of diagnosis via response confirmation as shown in by line 202. In embodiments, follow up questions and monitoring can be conducted through an application, such as a mobile interface for patient facing ease of use.

### Stage 3: Patient Cohort Identification

Stage 3 of patient intake process 200 comprises identifying patient group cohorts. Similar patient medical histories that share a diagnosis are associated enable comparison of clinical histories and results such that similar patients are more likely to receive similar care, and to enable identification of patients for drug/treatment trials. To determine patient cohorts parameters are determined based on the patient's medical history, diagnosis, developed treatment plan, and monitored results from the treatment plan. The global data lake is then searched to identify patients or existing patient cohorts with similar parameters and a report is provided to the clinician.

In grouping patients into cohorts, the system can generate correlations between treatment plans and patient factors. For example, if a certain drug is effective among a particular patient cohort, traits of patients within the cohort can be compared to traits of patients in a second cohort where the drug was ineffective. At stage 3 if a patient cohort is identified and a best outcome is determined based on clinician feedback then the diagnosis can be reconfirmed, and the treatment plan may be updated accordingly as shown by line 204. This iterative process allows for increasingly focused precision diagnosis.

In embodiments, patients can be grouped in cohort hierarchies wherein a patient may be in several patient cohorts depending on similarity of medical histories, including diagnosis. Over time, a patient's medical history may gradually be associated with more specific cohorts as the diagnosis is refined or broadened if uncertainties in the diagnosis of the patient arise. In embodiments, the elements of this process can be executed automatically or manually as directed by users of the system.

Referring to FIG. 4, a flow chart of search process 400 for automated search and aggregation of documentation from multiple data stores is depicted according to an embodiment. Search process 400 includes an automatic search based on the information currently available about a patient's medical history. This search produces a set of search paths which can indicate any cause-effect relationships between symptoms, medication, genetics, and other similar predicates that can be used to produce and/or retrieve medical information that is relevant to each of one or more patients. Medical information can include documentation, publications, test results, clinical guidelines, standards of care, or other data provided by search databases 106 in response to requests.

At 402 a patient's medical history is obtained and ingested such that patient data 128 is automatically deidentified, processed, and stored to a patient data lake 128. The ingestion process can use a natural language processing (NLP) system, such as the Apache CTAKES system. The NLP system can use word segmentation and optical character recognition to extract semantics and/or clinical named entities from text such as medical forms. Identified clinical named entities can be stored as labels in association with each record in patient data 128. In embodiments, the patient's medical history can be translated into a common language (such as English or Spanish) prior to being processed through NLP.

When ingesting patient data, the system can classify and sort incoming data as deidentified case data 132 or patient medical history 134 based on assigned labels. In some situations, patient data 128 can be stored and classified as either or both deidentified case data 132 and patient medical history 134 depending on the relevance of the data to the diagnosis and treatment plan of the patient. In embodiments, a user can manually classify data or change labels assigned to patient data 128.

In embodiments, the curation of a patient's medical history can be automated by the use of APIs enabling access to hospital systems or other health records systems to intake data. For example, an API can be provided by global data lake management engine 104 enabling external systems to provide patient medical history data. In addition, global data lake management engine 104 can access APIs provided by external medical records systems to retrieve patient data. NLP can be used to process the data, and the data can be structured into a format defined for the patient data lake. In embodiments, global data lake management engine 104 can provide one or more user interface elements enabling a user to enter data related to the patient's medical history after, for example, manually curating the patient's medical history from one or more originating points.

At 404, patient data 128 can be augmented with additional or related labels corresponding to medical concepts based on one or more medical ontologies. Augmentation can be conducted for each concept or named medical entity (such as symptom, diagnosis, or treatment) identified within patient data 128. For example, for each diagnosis within the patient's medical history a description can pulled from an integrated database of human maladies and their annotations, such as MALACARDS. The description of the diagnosis can be processed to produce multiple relevant labels, such as a “head pain” label for histories containing “headache” and “migraine.” Each search result can be tagged with at least one label. Each additional label can be stored in associated with records in patient data lake 128.

At 406 one or more search paths can be generated based on patient data 128 and/or the labels associated with patient data 128. Each search path can comprise one or more search terms (or sets of search terms), phrases, queries, predicates or other inputs that can be provided to search databases 106a-106N. Search paths can comprise, or be associated with a specific search database 106, or a group or classification of search databases 106. Search paths can also be general, such that search paths are processed through any number of search database 106 available to system 100. In embodiments, search paths can be generated in conjunction with one or more databases or systems describing relationships between medical concepts, such as MEDIKANREN, which is one autonomous reasoning agent (ARA) built upon the (National Center for Advancing Translation Sciences) NCATS Biomedical Data Translator Program. Such databases or systems can include proprietary ontologies.

Search paths can be generated based on one or more template predicates or questions to for searching within search databases 106. Example search paths can be generated by embodiments include (but are not limited to): which diseases or conditions are known to cause one or more symptoms present in patient data; which genes are known to correspond to the symptoms of diagnoses of the patient; which treatments are known to be effective for a diagnosis of the patient; which treatments are known to affect genes that are involved in a diagnosis of the patient. Search paths can be generated according to one or more relational programming languages such as Racket, miniKanren, Datalog, or the like. Each search path can then be processed by data lake association interface 110 and passed on to appropriate search databases 106. Labels can be assigned to each search path, and the search results received from corresponding search databases 106.

At 408, the labels tagged on each search result can be used to identify commonalities between the different search paths in order to group similar or equivalent search paths.

At 410 mutually exclusive search paths can be identified based on the assigned labels. For example, one search path labeled “hypertension” and another labeled “hypotension” could be identified as mutually exclusive due to the opposed labels.

At 412, the search paths and groups of search paths can be analyzed to determine if only one remains. Following the above example, in a situation where one search path is labeled “hypertension” and another is labeled “hypotension” if patient data indicates the patient has hypertension the hypotension search path can be discarded. Should only one search path or a group of search paths sharing commonalities remain at 414 the remaining search path(s) can be provided to search databases 106 to produce at least one search output. Search outputs can include relevant publications or other medical information. For example, each predicate within a search path may be associated with one or more supporting publications in a search database. Each supporting publication or other search result can be returned to the user, or automatically ingested into the patient data lake.

If multiple search paths remain after identifying commonalities and mutually exclusivities, at 416 the list of relevant labels can be used to automatically identify additional information needed to proceed. In embodiments, the identification of additional information can further include the identification of one or more suggested tests that may provide the additional information. For example, if two search paths differ in the expression (or not) of a particular gene, the provided search result can indicate one or more lab tests that can be performed to identify if the gene has been expressed or not. At 418 any identified tests can be performed and at 420 the results of such tests can be included in the patient's medical history before returning the search process is repeated by returning to 404.

In embodiments, machine learning can be implemented to develop the search iteration process. In such an embodiment, search iteration may begin somewhat manually in that users essentially conduct open searches for key terms and symptoms across a wide swath of data sources. Search terms and results can be gradually fed into a machine learning model that gradually identifies search terms and data sources with a greater likelihood of relevance for particular medical labels. Once sufficient training has been conducted, the search models can recommend search terms based on similar terms and research approaches that led to higher quality results in similar patient data lakes, the similarity of patient data lakes based on common terms and keywords in searches, and the similarity in patients' medical histories. Higher quality results can additionally come from increased confidence of association through NLP and scoring of patient reported outcomes.

Referring to FIG. 5, update process 400 for keeping data synchronized and updated is depicted according to an embodiment. As depicted, update process 400 comprises conducting automated searches 140 in response to the occurrence of one or more triggers. In embodiments, automated searches 140 can be triggered periodically, such as at twenty-four hour intervals. Automated searches 140 can also be triggered by the receipt of new patient data 128, or updated search results received from search databases 106. Automated searches 140 can be conducted by a process similar to process 400 discussed above with respect to FIG. 4.

For example, automated searches 140 can be triggered by the entry into patient data lake 126N of a new patient data item indicating a treatment for a patient. Global data lake management engine 104 can conduct one or more triggered search processes 140 through data lake association interface 110. Search databases 106 that have been associated with patient data lake 126N and are implicated by an automated search 140 can be searched and distilled data 142, which may pertain to the new treatment of this example, can be collected and incorporated into patient data 128N.

Similarly, search databases 106 can be monitored for updates. Where new medical research or other information is added to a relevant search database 106 distilled data 142 may be pulled from the search database 106N and assigned labels by data lake association interface 110. This labeled distilled data 142 can then be incorporated into any relevant patient data lake 126.

Update process 400 can enable patient data lake 126 to be kept up-to-date with medical information based on updated patient medical history and medical advancements available in search databases 106.

In embodiments, update process 300 can be applied to patient cohorts, patient data lakes, and the global data lake within global data lake management engine 104 depending on the level of incoming information and preferences of a user. For example, if new research is published that applies to kidney patients, the global data lake can ingest the new publication, use NLP to identify labels relevant to the new publication, and match the new publication to relevant patient data lakes or patient cohorts, and score the confidence of the match. When a new search is triggered, patients that have similar profiles/data lakes can all be updated as necessary. In embodiments, the update process can be triggered on a scheduled basis (e.g., daily), or whenever new data from a patient data lake indicates the presence of a new treatment or identifies new research.

This two-way update process provides significant advantages over conventional systems that fail to associate individual patients with new medical research that is directly relevant to the patient's case. The relevancy of data can be determined by the label associations assigned to each of patient data 128 and medical information within search databases 106. This association greatly reduces the clinician's burden by directly recommending new approaches specific to their patient's symptoms, diagnosis, past treatments, and current treatment plan rather than inundating the clinician with all medical research that might apply to the patient based on broader categorization, such as only the clinician's diagnosis.

In embodiments, various reports can be automatically produced at different stages of the patient intake process. A patient medical history report can be produced at any time either manually or generated by a model capable of machine learning. During the diagnosis confirmation and verification stage a suggested diagnoses report may be generated including color coding consistent with confidence levels associated with each diagnosis. During the treatment development and monitoring stage, a report of suggested treatments may be produced including annotations according to criteria such as difficulty of implementing the treatment plan or risk to the patient. In embodiments, these annotations can be based on industry standard tables. Potential annotations include, known or potential undesirable interactions being highlighted, presenting a table of intervention options that have not been utilized by the patient previously, a synopsis of justification for care, and literature citations embedded within the report for optional further review by the clinician.

Patient reports can be automatically generated using NLP and comprise comprehensive summaries of a patient's entire medical history. In embodiments, reports can be manually generated by a user by pulling patient data based on labels assigned during patient data ingestion and searching. Sample reports and user interfaces are depicted in FIGS. 6A-6D, according to embodiments. In embodiments, each patient's unique identifier can be used to produce and request a report without associating the report with PII. In embodiments, deidentified case data of any recent diagnosis and medical history of the patient can guide a precision diagnosis based on patient specific characteristics and matching of symptoms to a likely disease. On produced reports, deidentified case data can provide a high-level summary for a clinician to efficiently review without having to dig through potentially years of medical tests and failed treatments. Reports and any other outputs of system 100 can be provided via user interfaces, or in one or more electronic file formats including unstructured text, structured text (extensible markup language (XML), JavaScript Object Notation (JSON) etc.), Portable Document Format (PDF), and/or as API accessible databases/data stores.

In embodiments, patient medical history 134 presented on reports can provide clinicians with a digestible view of a patient's detailed medical history, including a patient's medication history. Levels of detail can be presented to the clinician in such a way as to quickly convey basic information while enabling deep dives into research or past treatments should the clinician be interested. These levels of detail for patient medical histories can be presented using thumbnails, hover boxes, color coding schemes, and embedded linkable keywords. In embodiments, reports can include indicators of the patient's medical history 134 relative to one or more standards of care determined to be relevant based on labels associated with patient data 128.

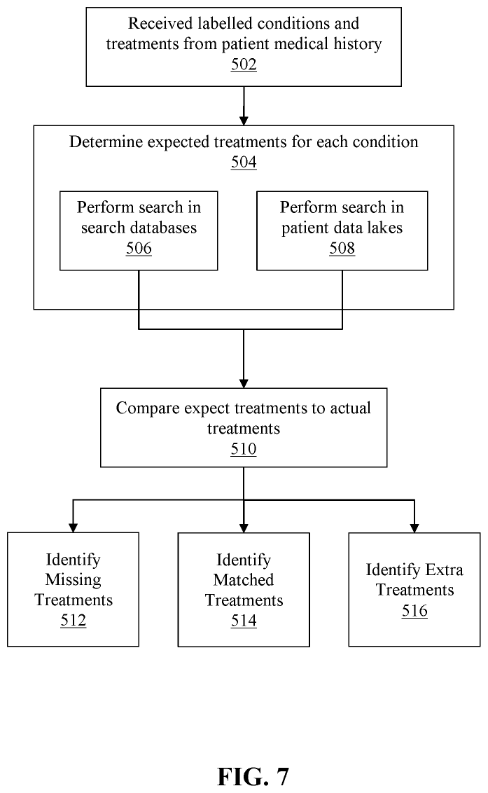

FIG. 7 is a flowchart depicting a method 500 for displaying patient data, according to an embodiment. Patient medical history including labelled conditions and treatments can be received at 502. Patient medical history can be received, ingested, and labelled per methods discussed herein.

At 504, for each condition identified in the patient medical history, expected or potential treatments can be retrieved. At 506 a search can be performed in one or more search databases 106 to determine known treatments for a condition. For example, search databases mapping diseases to drugs (such as DRUGBANK) can be consulted to determine the identities of one or more drugs known to treat the condition. At 508, global data lake management engine 104 can be queried to determine any, all, or the most common treatments associated with patient data lakes 128 that include a label corresponding to the condition. In embodiments, only data lakes associated with patients in a related cohort are consulted. The expected treatments determined based on search databases and data lakes can be combined or aggregated into a single set or list of expected treatments.

At 510, the expected treatments can be compared to the actual treatments determined from the patient medical history. At 512, missing treatments can be identified. Missing treatments can be treatments in the set of expected treatments that are not within the set of actual treatments. At 514 matched treatments can be identified. Matched treatments can be treatments in the set of expected treatments that have a corresponding match in the patient medical history. At 516, extra treatments can be identified. Extra treatments can be treatments in the set of actual treatments that do not correspond to treatments for any of the conditions in the patient's medical history. In embodiments, missing, matched, and extra treatments can be displayed on a user interface with color-coding or other identifying indicia.

In embodiments, treatments can be considered matched where an actual treatment is related to a matched treatment in one or more ontologies, such as an ontology providing relationships between brand name drugs and generic equivalents. In embodiments, various degrees of match can be assigned based on, for example, a dosage of a medicament based on the patient's demographic data (height, weight, or age). Treatments can also be matched where a patient is not currently undergoing an expected treatment but has in the past.

Reports generated based on method 500 can provide clinical support for comparing current patient treatments with practice guidelines or standards of care. The reports can enable clinicians to quickly identify missing treatments, or extra treatments that may be candidates for cessation—for example where a patient has recovered from a condition but has still be prescribed a treatment. Extra treatments can also indicate one or more conditions not properly identified in patient medical history, for example where the condition was not previously entered into the medical history, or where processing or translation errors have occurred.

In embodiments, a patient's compliance with a treatment plan and the effectiveness of the treatment plan can be tracked. Successful treatment or negative side effects from the treatment plan can then be compared to other patients with a similar medical history or within the same patient cohort to refine the given diagnosis. This updated treatment data can feed back into the patient data lake and spur update processes for similar patients.

Identifying connections between similar patients and continued updates to medical developments can facilitate faster identification of patients that might be suffering from rare diseases and aggregation of any similar rare disease cases. Patients suffering from rare diseases can then be better matched with clinical trials and drug targeting to develop treatment plans.

The NLP used in report generation can follow any of a number of approaches depending on the source of the medical data, each of which can require different levels of integration. For example, if the medical data comes from a source incorporating an API, retrieval of information can involve making an API call to request medical data for a desired field (patient name, medications delivered to the patient, etc.).

In another approach, data (or, database) mapping can be implemented to homogenize multiple data sets. Data mapping is the process of matching fields from multiple datasets into a schema, or centralized database. Data mapping can facilitate the migration, ingestion, processing, and management of data.

In yet another approach, the system can receive the identification of different sections (patient summary, diagnosis, etc.) of a PDF or another generic file format used by a healthcare provider. This identification can be performed manually by a user or automated, in embodiments. This approach can leverage consistent file templates and forms in use across a healthcare provider's network. Regular expression processing can then be implemented to automatically distinguish the different sections of similarly arranged files based on the manually identified example.

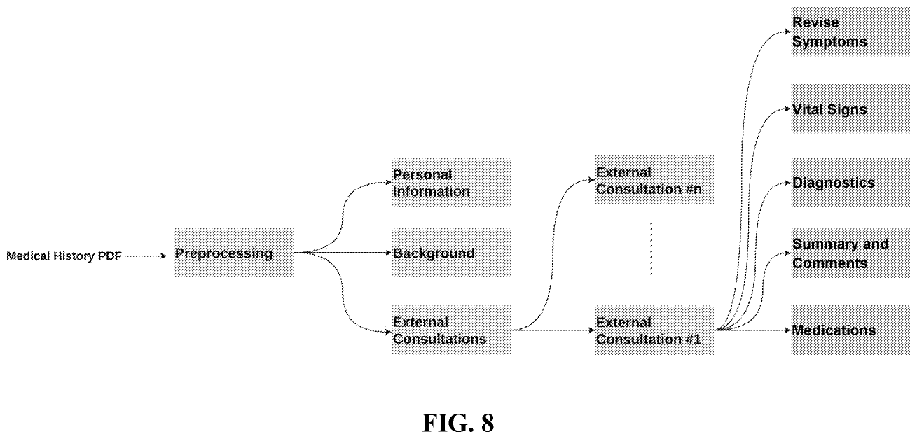

Alternatively, as shown in FIG. 8, a neural network can be trained using machine learning to distinguish various hierarchal sections of a file. As depicted, a medical history PDF can be broken into more specific blocks of classifications. For example, a neural network can identify a portion of a patient's medical history document that represents external consultations and then can separate that section into each external consultation the patient has received. From there, the neural network can identify and label key elements of the patient's medical history, such as diagnostics and medications given. In embodiments, a neural network can be taught to identify classification blocks by analyzing document characteristics, including font size, spacing within the file, and identification of keywords. In other embodiments, a neural network may be trained using examples generated manually.

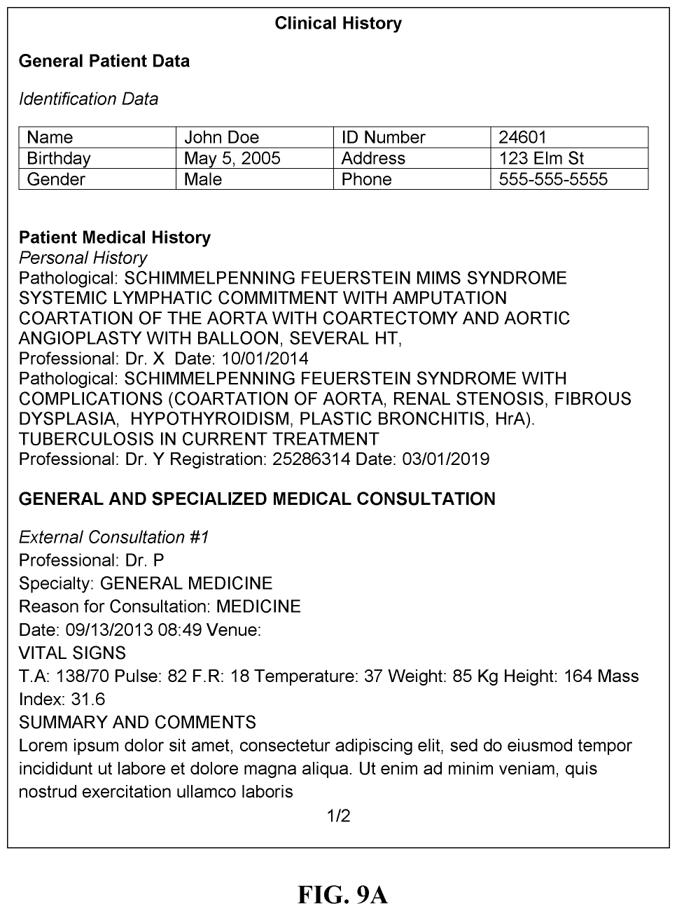

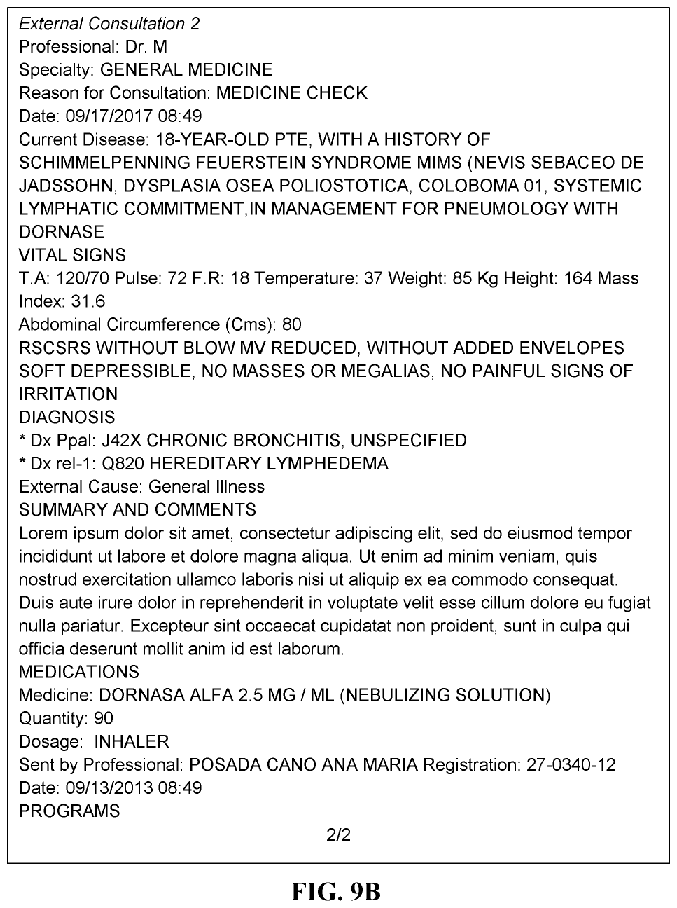

An example clinical history document is provided at FIGS. 9A and 9B. At the preprocessing step, a first set of markers can be identified, for example:

| | |
|---|---|
| Marker #1 | /\hat{ }IDENTIFICATION DATA$/ |
| Marker #2 | /\hat{ }HISTORY$/ |
| Marker #3 | /\hat{ }EXTERNAL CONSULATIONS$/ |
| Marker #4 | /\hat{ }PROGRAMS$/ |

While the markers depicted herein are based on the text content, other attributes of the text can be used to distinguish markers, for example font, font size, style attributes, or other metadata. The block of medical history between markers 1 and 2 identified as personal information and discarded. The block of medical history between markers 2 and 3 can identified as patient background information and processed by an NLP model. The block of medical history between markers 3 and 4 can be identified as external consultations and further broken down.

One or more markers for identifying individual external consultations can be used, for example:

| | |
|---|---|
| Marker #5 | /\hat{ }EXTERNAL CONSULATION #[0-9] [1-9] [0-9] [1-9]$/ |

Each individual external consultation block can then be broken down into individual sections based on additional markers such as:

| | |
|---|---|
| Marker #6 | /\hat{ }REVISED SYMPTOMS$/ |
| Marker #7 | /\hat{ }VITAL SIGNS$/ |
| Marker #8 | /\hat{ }DIAGNOSTICS $/ |
| Marker #9 | /\hat{ }SUMMARY AND COMMENTS$/ |
| Marker #10 | /\hat{ }MEDICATION$/ |

An existing technical problem in handling patient medical records and histories is an inability to efficiently process large quantities of information that are stored differently across healthcare systems. The present disclosure addresses this concern by dynamically applying NLP labelling using ontologies upon receiving new patient data. This labeling can result in faster search times for the computer because the search function does not need to process entire texts for each search request. Additionally, NLP labeling simplifies the process of associating similar patients and search databases, dramatically speeding up the update process when new information is recognized.

Labeling data further reduces computational overhead associated with making database calls to the various search databases. Preprocessing of search paths using labels requires fewer access calls to MEDIKANREN or other databases further improving search efficiency and system resource allocation.

Report generation can require significant resources and time when handling and sorting through large amounts of patient data. The present disclosure addresses this issue by automatically converting patient data records to standard formats thereby enabling generation of reports based on known fields. Additionally, dynamic labeling and classification of patient data, such as past diagnosis, allows the system to rapidly pull critical information for the clinician. This labeling and classification of data are facilitated by the update process that consistently reassess the value and confidence score of patient data based on the most recent and relevant medical research and treatment plans.

It should be understood that the individual steps used in the methods of the present teachings may be performed in any order and/or simultaneously, as long as the teaching remains operable. Furthermore, it should be understood that the apparatus and methods of the present teachings can include any number, or all, of the described embodiments, as long as the teaching remains operable.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

GENECARDS (accessible at genecards.org) and MALACARDS (accessible at malacards.org), are elements of the GeneCards Suite Products published by the WEIZMANN INSTITUTE OF SCIENCE and LIFEMAP SCIENCES. DRUGBANK is a database containing information on drugs and drug targets maintained by the UNIVERSITY OF ALBERTA. GOOGLE SCHOLAR is a searchable database of research articles, theses, books, abstracts, and court opinions from a variety of disciplines published by GOOGLE LLC. DISEASE ONTOLOGY is a formal ontology of human disease hosted at the INSTITUTE FOR GENOME SCIENCES at the UNIVERSITY OF MARYLAND SCHOOL OF MEDICINE. CTAKES is a natural language processing system for extraction of information from electronic medical record clinical free-text published by THE APACHE SOFTWARE FOUNDATION. CLINICALTRIALS.GOV is a resource for medical studies and clinical trial results provided by the U.S. NATIONAL LIBRARY OF MEDICINE. MEDIKANREN tool combining the miniKanren family of programming languages for relational programming with a database describing relationships between medical concepts and a graphical user interface to simplify data exploration and common queries published by researchers at the UNIVERSITY OF ALABAMA AT BIRMINGHAM. All copyrights and trademarks are the properties of their respective owners.

For purposes of interpreting the claims, it is expressly intended that the provisions of 35 U.S.C. § 112(f) are not to be invoked unless the specific terms “means for” or “step for” are recited in a claim.

1. A system for coordinating patient medical histories for rare-disease identification, comprising:
an interface configured to search a plurality of search databases including medical data;
a global data lake configured to include a plurality of patient data lakes, each patient data lake configured to store patient data of at least one patient of a plurality of patients;
at least one processor having a memory operably coupled to the processor; and
the memory further comprising instructions that, when executed on the at least one processor, cause the at least one processor to, for each patient of the plurality of patients:
receive a medical history for the patient, the medical history comprising at least one record;
label each record of the medical history according to at least one medical ontology;
generate a plurality of search paths based on the labeled records;
label the plurality of search paths according to the at least one medical ontology;
identify commonalities between the plurality of search paths;
identify pairs of search paths of the plurality of search paths that are mutually exclusive;
selectively request medical data relevant to the patient based on the identified commonalities between the plurality of search paths and the identified pairs of the plurality of search paths that are mutually exclusive from at least one of the plurality of search databases, and
update the patient data to include the medical data.

2. The system of claim 1, wherein at least one of the search paths includes a symptom of the patient and the medical data is a condition corresponding to the symptom of the patient.

3. The system of claim 1, where in at least one of the search paths includes a diagnosis of the patient and the medical data is a treatment associated with the diagnosis of the patient.

4. The system of claim 1, wherein the instructions are further configured to cause the processor to:
identify at least one item of medical information selected to differentiate the plurality of search paths that are mutually exclusive; and
in response to receiving the at least one item of medical information, selecting a search path of the search paths that are mutually exclusive and requesting the medical data from at least one of the plurality of search databases based on the selected search path.

5. The system of claim 4, wherein the at least one item of medical information is a result of a medical test.

6. The system of claim 5, wherein the medical test is a test to determine whether a gene is expressed in the patient.

7. The system of claim 1, wherein the instructions are further configured to cause the processor to, for each patient of the plurality of patients:
augment at least one record of the medical history by requesting medical data relevant to the at least one record from at least one of the plurality of search databases.

8. The system of claim 1, wherein the instructions are further configured to cause the processor to, for each patient of the plurality of patients:
identify at least one label associated with a condition of the patient;
identify at least one label associated with a treatment of the patient;
determine a plurality of expected treatments associated with the condition by requesting medical data relevant to the at least one label associated with the condition of the patient from at least one of the plurality of search databases; and
produce an output indicating any expected treatments of the plurality of expected treatments that do not have at least one label associated with a treatment of the patient.

9. The system of claim 8, wherein the output further indicates whether the treatment of the patient is associated with at least one expected treatment of the plurality of expected treatments.

10. The system of claim 1, wherein the instructions are further configured to cause the processor to:
selectively request medical data relevant to the patient based on the plurality of search paths from at least one second patient data lake of the plurality of patient data lakes.

11. The system of claim 1, wherein the instructions are further configured to cause the processor to:
periodically monitor at least one of the plurality of search databases for updated medical data.

12. A computer-implemented method for coordinating patient medical histories for rare-disease identification, comprising:
receiving, at a computing device comprising a memory and a processor, a medical history for a patient, the medical history comprising at least one record;
labelling each record of the medical history according to at least one medical ontology;
storing each labeled record in a patient data lake configured to electronically store patient data of the patient;
generating a plurality of search paths based on the labeled records;
labelling each search path of the plurality of search paths according to the at least one medical ontology;
identifying commonalities between the plurality of search paths;
identifying pairs of the plurality of search paths that are mutually exclusive;
selectively requesting medical data relevant to the patient based on the identified commonalities between the plurality of search paths and the identified pairs of the plurality of search paths from at least one search database communicably coupled to the computing device; and
updating the patient data lake to include the medical data.

13. The method of claim 12, wherein at least one of the search paths includes a symptom of the patient and the medical data is a condition corresponding to the symptom of the patient.

14. The method of claim 12, wherein at least one of the search paths includes a diagnosis of the patient and the medical data is a treatment associated with the diagnosis of the patient.

15. The method of claim 12, further comprising:
augmenting at least one record of the medical history by requesting medical data relevant to the at least one record from the at least one search database.

16. The method of claim 12, further comprising:
identifying at least one item of medical information selected to differentiate the plurality of search paths that are mutually exclusive; and
in response to receiving the at least one item of medical information, selecting a search path of the search paths that are mutually exclusive and requesting the medical data from at least one search database based on the selected search path.

17. The method of claim 16, wherein the at least one item of medical information is a result of a medical test.

18. The method of claim 17, wherein the medical test is a test to determine whether a gene is expressed in the patient.

19. The method of claim 12, further comprising:
identifying at least one label associated with a condition of the patient;
identify at least one label associated with a treatment of the patient;
determine a plurality of expected treatments associated with the condition by requesting medical data relevant to the at least one label associated with the condition of the patient from the at least one search database; and
produce an output indicating any expected treatments of the plurality of expected treatments that do not have at least one label associated with a treatment of the patient.

20. The method of claim 19, wherein the output further indicates whether the treatment of the patient is associated with at least one expected treatment of the plurality of expected treatments.

21. The method of claim 12, further comprising selectively requesting medical data relevant to the patient based on the at least on search path from at least one second patient data lake.

22. The method of claim 12, further comprising periodically monitoring the at least one search database for updated medical data.

23. A method for consolidating and presenting a medical history of a patient, comprising:
receiving the medical history of the patient, the medical history including target patient data;
labeling, via a natural language processing system, the target patient data based on medical ontologies;
storing the target patient data in one of a plurality of patient data lakes;
comparing the target patient data with existing patient data from at least one of the plurality of patient data lakes to identify similarities based on at least one of symptoms, diagnosis, or treatment plans;
classifying a relevance of the target patient data based on the labels and the identified similarities; and
producing a report of the target patient data classified as most relevant.

24. A system for associating patients, comprising:
a global data lake configured to include a plurality of patient data lakes each storing patient data of at least one patient;
at least one processor having a memory operably coupled to the processor; and
the memory further comprising instructions that, when executed on the at least one processor, cause the at least one processor to, for each of the plurality of patients:
label the patient data using natural language processing based on medical ontologies,
generate a diagnosis report confirming a previous diagnosis or providing a first alternative diagnosis,
receive diagnosis feedback from a user, the diagnosis feedback including a selected diagnosis,
generate a treatment plan based on the selected diagnosis,
receive treatment plan feedback from the user, the treatment plan feedback including monitored patient outcomes as a result of the treatment plan,
update the diagnosis report to the selected diagnosis or provide a second alternative diagnosis based on the labeled patient data, the diagnosis feedback, and the treatment plan feedback.

25. A method for consolidating and presenting a medical history of a patient, comprising:
receiving the medical history of the patient, the medical history including target patient data;
labeling, via a natural language processing system, the target patient data based on medical ontologies;
storing the target patient data in one of a plurality of patient data lakes;
comparing the target patient data with existing patient data from at least one of the plurality of patient data lakes to identify similarities based on at least one of symptoms, diagnosis, or treatment plans;
classifying the relevance of the target patient data based on the labels and the identified similarities; and
producing a report of the target patient data classified as most relevant.
